(19) United States
(12) Patent Application Publication
(10) Pub. No.: US 2021/0317217 A1
(43) Pub. Date: Oct. 14, 2021

(54) HUMANIZED ANTI-VEGFR2 SINGLE-CHAIN ANTIBODY AND USE THEREOF

(71) Applicant: ZHEJIANG BLUE SHIELD PHARMACEUTICAL CO., LTD., Shaoxing (CN)

(72) Inventors: Zhigang GUO, Shaoxing (CN); Binghui SHEN, Shaoxing (CN); Laosheng WU, Shaoxing (CN)

(21) Appl. No.: 17/271,909

(22) PCT Filed: Apr. 29, 2019
(86) PCT No.: PCT/CN2019/084875

§ 371 Date: Feb. 26, 2021

(30) Foreign Application Priority Data
Aug. 30, 2018 (CN) 201811003017.7

(51) Int. Cl. C07K 16/28; A61P 35/00

(52) U.S. Cl. CPC C07K 16/2863; A61P 35/00; C07K 2317/76; C07K 2317/622; C07K 2317/565; C07K 2317/24

(57) ABSTRACT

The present invention belongs to the field of antibody drugs, and relates to a humanized anti-VEGFR2 single-chain antibody and use thereof. The single-chain antibody comprises a heavy chain variable region set forth in SEQ ID No. 1 and a light chain variable region set forth in SEQ ID No. 2, wherein the heavy chain variable region and the light chain variable region are connected via a flexible peptide with an amino acid sequence set forth in SEQ ID No. 3. The present invention also discloses use of the antibody in preparing a product for inhibiting tumor growth. The antibody disclosed herein can be used as a medicament for clinical use in indications caused by neovascularization.

### TECHNICAL FIELD

The present invention belongs to the field of antibody drugs, and relates to a fully humanized anti-VEGFR2 single-chain antibody and use thereof.

### BACKGROUND

Tumor blood vessels provide enough oxygen and nutrition for the generation and development of tumors, and targeting tumor angiogenesis can achieve the therapeutic effect of starving tumors. However, the molecular regulation mechanism of tumor angiogenesis is so complicated that the mediation of a plurality of growth factors, receptors and signaling pathways is required.

In the angiogenesis process, vascular endothelial cell growth factors (VEGFs) play an important role in strongly stimulating the proliferation of vascular endothelial cells. The receptors to which VEGF binds include VEGFR1 (also known as Flt-1) and VEGFR2 (also known as Flk-1). VEGFR1 and VEGFR2 are in the type III receptor tyrosine kinase family. Of which, extracellular region consists of 7 immunoglobulin-like domains and includes a ligand binding domain and a receptor dimerization domain, middle region comprises a cell transmembrane domain, and intracellular region comprises a tyrosine kinase domain. VEGFR2 mediates a variety of effects of VEGF including endothelial cell proliferation, vascular proliferation and infiltration, whereas VEGFR1 does not appear to be directly involved in endothelial cell proliferation and vascular proliferation. The binding of VEGF dimer to VEGFR2 induces receptor dimerization and phosphorylation of tyrosine residues in the intracellular tyrosine kinase domain, thereby activating downstream signaling pathways, including activation of phospholipase C, increase of intracellular Ca²⁺ concentration and the like, triggering events including the proliferation and survival vascular endothelial cells, cytoskeletal rearrangement, cell migration, gene expression and the like, ultimately leading to vascular proliferation.

Bevacizumab, an inhibitor against VEGF, is a recombinant humanized monoclonal antibody against VEGF developed by Genentech, which consists of 93% human and 7% mouse sources. Bevacizumab was approved for marketing in the United States by FDA on Feb. 26, 2004, being the first approved drug to market in the United States to inhibit tumor angiogenesis. Global sales of bevacizumab were $556 million in 2004 and up to $7.037 billion in 2013. Ramucirumab, an inhibitor against VEGFR2, was developed and successfully marketed by Eli Lilly and Company following its acquisition of ImClone's IMC-1121B project. Ramucirumab is a fully human IgG1 monoclonal antibody against VEGFR2 that specifically binds to KDR/VEGFR2. The drug was marketed in the United States in May 2014 for use in advanced or metastatic gastric cancer or adenocarcinoma of esophagogastric junction.

### SUMMARY

The present invention is intended to provide a humanized anti-VEGFR2 antibody and use thereof.

In the method of the present invention, a high-capacity high-diversity antibody library is constructed, and from which antibodies that bind to VEGFR2 and can block their binding to ligand VEGF are screened, and the resulting antibodies have cell viability.

The present invention provides a humanized anti-VEGFR2 antibody.

Provided is an anti-VEGFR2 single-chain antibody, comprising a heavy chain variable region and a light chain variable region, wherein the heavy chain variable region and the light chain variable region are connected via a flexible peptide with an amino acid sequence set forth in SEQ ID NO. 3.

The heavy chain variable region can specifically be a polypeptide set forth in SEQ ID NO. 1 of the sequence listing.

The light chain variable region can specifically be a polypeptide set forth in SEQ ID NO. 2 of the sequence listing.

The monoclonal antibody disclosed herein is fully humanized.

CDR1, CDR2 and CDR3 in the heavy chain variable region are sequentially amino acid residues at positions 26-38, amino acid residues at positions 53-69 and amino acid residues at positions 102-107 from the N terminus of sequence 1 in the sequence listing; CDR1, CDR2 and CDR3 in the light chain variable region are sequentially amino acid residues at positions 24-35, amino acid residues at positions 51-58 and amino acid residues at positions 93-101 from the N terminus of sequence 2 in the sequence listing.

The present invention also discloses use of the anti-VEGFR2 single-chain antibody in preparing a product for inhibiting tumor growth.

The tumor growth is reflected in an increased volume of the tumor and/or an increased mass of the tumor.

The sequences of the antibody gene variable region involved in the present invention can construct a full-length antibody molecule as a medicament for clinical use in indications caused by neovascularization.

### BRIEF DESCRIPTION OF THE DRAWINGS

FIG. 1 is an SDS-PAGE electrophoretogram of VEGFR2-specific single-chain antibody purification.

FIG. 2 is a graph showing EC50 for different concentrations of ligands of VEGFR2-specific antibodies.

FIG. 3 is a graph showing the relationship between tumor volume and time of administration (AVR2 vs. Avastin).

FIG. 4 is a graph showing the solid tumor volume change after administration.

### DETAILED DESCRIPTION

The following examples are intended to facilitate a better understanding of the present invention, but do not limit the present invention. Unless otherwise stated, the methods in the following examples are conventional methods. Unless otherwise stated, the test materials used in the following examples are purchased from conventional biochemical reagent stores. In quantitative tests of the following examples, three replicates are set up and the results are averaged.

### Example 1: Discovery of VEGFR2-Specific Antibodies

I. Construction of Natural Humanized Single-Chain Antibody Library

Peripheral blood was obtained from the volunteers with informed consent, from which lymphocytes were isolated and total RNA was extracted. The total RNA was reversely transcribed to obtain cDNA. The heavy chain and light chain variable regions (VH and VL) of the antibody were PCR amplified with degenerate primers using cDNA as a template. The PCR products were subjected to 1.5% agarose gel electrophoresis, and

VH DNA and VL DNA were isolated and ligated by overlap PCR with the gene encoding the flexible peptide (SEQ ID NO. 3) as a single-chain antibody (scFv) gene. The single-chain antibody genes from different volunteers were mixed in equal amounts, grouped and cleaved by restriction enzyme, followed by 1.5% agarose gel electrophoresis. DNA fragments were isolated and ligated to the phagemid vector plasmid that has been cleaved by the same restriction enzyme, and the ligated phagemid vector plasmid was electrically transferred into E. coli to obtain a phage single-chain antibody library.

II. Screening for VEGFR2 Humanized Monoclonal Antibodies

1. Phage Display and Panning of Antibody Library

100-fold library capacity of bacterial solution of the above humanized VH and VL single-chain antibody library was seeded in 900 mL of 2YT-AG medium (containing 100 μg/mL ampicillin and 2% glucose), which was then cultured at 37° C. and 250 rpm until OD600 was 0.5-0.6. Then helper phages with 100-fold cell density were added for infection for 0.5 h, and the bacteria were collected by centrifugation. The cells were resuspended in 900 mL of 2YT-AK medium (containing 100 μg/mL ampicillin and 50 μg/mL kanamycin), and cultured at 30° C. and 250 rpm overnight.

The culture from the previous step was centrifuged at 10,000 rpm and 4° C. for 20 min, and the supernatant was collected, added with ¼ volume of PEG/NaCl, mixed well, and left to stand on ice for 2 h. The mixture was centrifuged at 10,000 g and 4° C. for 25 min, and the supernatant was discarded. The centrifuge tube was inverted on sheet paper to remove the liquid. The phage precipitates were resuspended in 3 mL of pre-cooled 1×PBS and centrifuged at 12,000 g and 4° C. for 5 min, and the supernatant was transferred to a new 15 mL centrifuge tube to obtain the first round of initial phages.

VEGFR2-Fc was used as an antigen to coat an immune tube, which was then blocked with 3% M-PBS. Then 100× library capacity of the first round of initial phages was added for antibody-antigen binding, and the unbound phages were washed away with PBST. The phages were eluted with 0.6 mL of triethylamine for 5 min, and equilibrated with 0.6 mL of 1 M Tris-HCl (pH 7.4). The eluted phages were used to infect TG1, the eluted products were amplified, and the phages were precipitated and purified with PEG/NaCl for next round of screening. A total of 3-4 rounds of enrichment screening on the phage library were performed. The antigen amount was decreased sequentially, and the washing intensity was increased sequentially. The eluted products in each round were measured for titer.

2. Induction of Expression of Monoclones and ELISA Screening

The panned bacterial solution with limited dilution was coated on a plate, and cultured overnight. Monoclones were selected and cultured overnight in a 96-well deep well plate containing 2YT-AG medium at 0.5 mL/well. The overnight culture was transferred to a 96-well deep well plate containing 2YT-AG culture medium at 0.5 mL/well in a ratio of 1:10, and cultured until OD600 was 0.5-0.6. Then helper phages were added for infection at 37° C. for 15 min, and cultured at 37° C. for 45 min. The bacteria were collected by centrifugation at 4000 g, resuspended in 2YT-AK culture medium (containing 100 μg/mL ampicillin and 50 μg/mL kanamycin), induced overnight at 30° C., and centrifuged on the next day. The supernatant was transferred to a clean 96-well deep well plate to obtain monoclonal phage samples.

VEGFR2-Fc was used as an antigen to coat a 96-well microplate, and 50 μL of monoclonal phage samples was added to each well after the microplate was blocked, followed by incubation for 1.5 h at 37° C. Then 300 μL of PBST was added to each well and shaken for 5-10 s before the solution was discarded, repeating for 3-5 times. After that, 100 μL of PBST diluent of anti-M13-HRP antibody was added to each well, and incubated at 37° C. for 1 h. Then 300 μL of PBST was added to each well, and shaken for 5-10 s before the solution was discarded, repeating for 5 times. 50 μL of TMB color developing solution was added to each well to develop color for 3-10 min (the specific color developing time depends on the color developing speed), and then 50 μL of 1 M H₂SO₄ was added to each well to stop developing. OD450 values were determined using a microplate reader. ELISA positive samples were selected according to the ELISA data of the monoclonal phages. The above overnight culture broth in the 2YT-AG culture medium of the 96-well deep well plate was taken for sequencing analysis, and the sequence of the unique monoclonal antibody was obtained and set forth in SEQ ID NO. 6 (consisting of SEQ ID NOs. 1, 3 and 2).

### Example 2: Expression of VEGFR2 Antibodies

I. Construction of Recombinant Plasmid

1. DNA molecule encoding VEGFR2-specific antibody was PCR amplified using a primer pair of SCFV-F and SCFV-R to obtain PCR amplification product.

| (SEQ ID NO. 4) | |
|---|---|
| ScFv-F: | CTACGGCAGCCGCTGGATTG |
| (SEQ ID NO. 5) | |
| ScFv-R: | CTCGAGGCCTGAGGAGACGGTGAC |

2. The PCR amplification product obtained in step 1 was cleaved with restriction enzymes Nco I and Xho I, and the cleaved product was isolated.

3. The pET28B plasmid (purchased from Novagene) was cleaved with restriction enzymes Nco I and Xho I, and the vector backbone was isolated.

4. The cleaved product from the step 2 was connected to the vector backbone in the step 3 to obtain a recombinant plasmid pET28B-ScFv.

II. Acquisition of Recombinant Strain

The recombinant plasmid pET28B-ScFv was transformed into BL21 competent cells to obtain a recombinant strain.

### Example 3: Large-Scale Preparation and Purification of VEGFR2-Specific Antibodies

1. Bacteria stored in a refrigerator at −80° C. were streaked on a Kan-resistant plate, and cultured at 37° C. overnight (about 15 h). Monoclones were selected and seeded in 3 mL of Kan-resistant liquid LB medium, and cultured with shaking at 37° C. and 200 rpm overnight for about 15 h. 1 mL of bacterial solution was seeded in 100 mL of fresh Kan-resistant liquid culture medium (1:100), and cultured with shaking at 37° C. and 200 rpm. When OD600 of the bacterial solution reached 0.6, IPTG mother solution was added to make the final concentration of 0.5 mmol/L. The mixture was cultured with shaking at 30° C. and 200 rpm for 3 h, and centrifuged at 4° C. and 1000 rpm for 10 min to collect bacteria. The bacteria were resuspended in PBS, and centrifuged under the same conditions to collect bacteria, which were directly used for bacteria lysis.

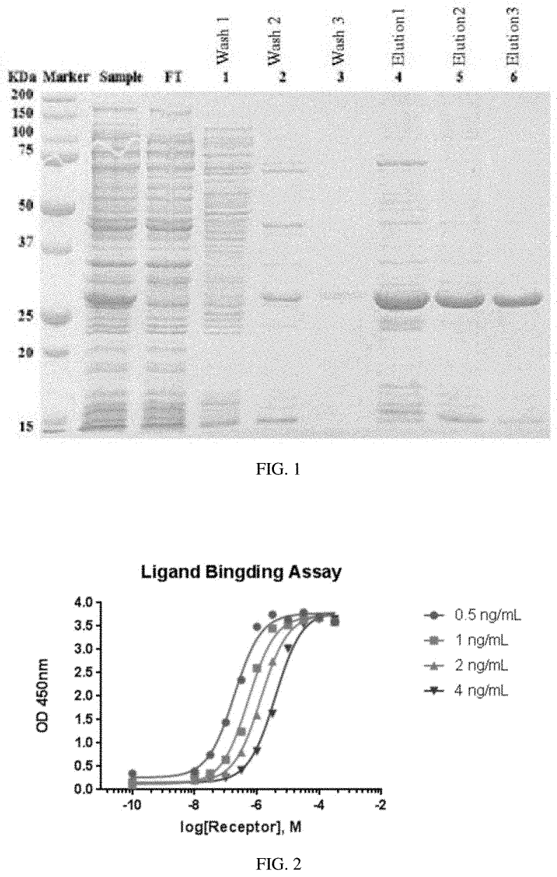

2. Preparation of sample (using a Sangon Ni-TED pre-loaded gravity column, 1 mL): the host cell debris was removed by centrifugation or the like, then passed through a 0.45 μm microfiltration membrane and diluted appropriately with a binding buffer. Water washing: the resin was washed with 5-10 fold column volume of pure water at 50-150 cm/h to remove ethanol. Equilibration: the medium was equilibrated with 5-10 fold column volume of a binding buffer at 150-600 cm/h, so as to ensure that the components and the pH of the solution in the medium are consistent with those of the sample. Sample loading: the sample was centrifuged, filtered (0.45 μm) and loaded at a low flow rate. If the column height is 20 cm, the recommended flow rate is ≤150 cm/h, and if the column volume is 1 mL, washing impurities: impurities were washed with 10-20 fold column volume of a washing buffer at 150 cm/h to remove non-specifically adsorbed impure proteins, and the washing buffer was collected for subsequent analysis. Elution: elution was performed with 5-10 fold column volume of elution buffer at a low flow rate, and the eluent was collected, detected with SDS-PAGE (shown in FIG. 1), and concentrated and desalted using an ultrafiltration tube. The target protein was stored at −20° C.

### Example 4: Binding of VEGFR2 Antibodies to Ligand

Ligands (VEGFR2 antigens) at different concentrations were immobilized on a microplate. Then, VEGFR2 antibodies at different concentrations were added and incubated at 37° C. for 2 h. The unbound antibodies were washed away. The antibodies that bind to ligands were detected using anti-human IgG-HRP (from Invitrogen). The results are shown in FIG. 2. The results showed that the VEGR2 antibody binds strongly to ligand.

### Example 5: Treatment of Breast Cancer with VEGFR2

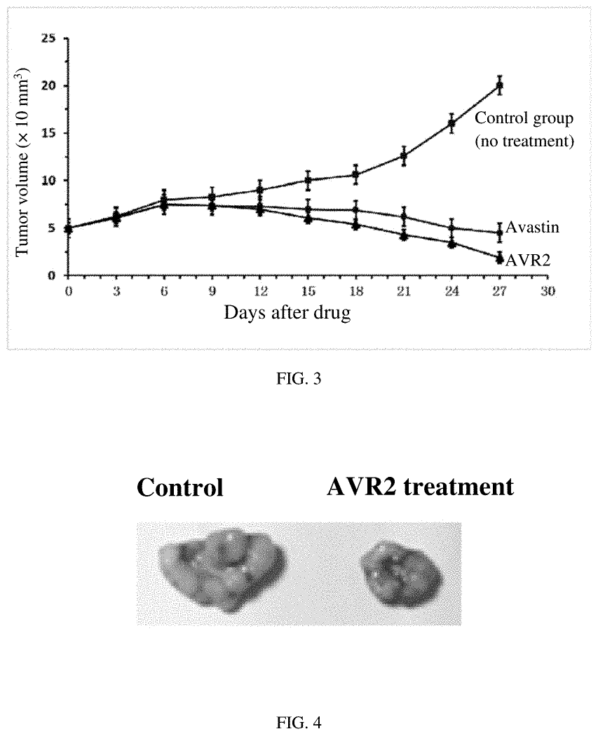

Nude mice were purchased from Model Animal Research Center. Nude mice were inoculated with breast cancer cells, and 2 weeks later were injected with VEGFR2 antibody and control drugs (FIG. 3). The results showed that VEGFR2 can effectively inhibit tumor growth, and its effect is superior to Avastin under the test conditions. FIG. 4 shows the comparison of tumor size in the treated group and the control group.

What is claimed is:

1. A humanized anti-VEGFR2 single-chain antibody, comprising a heavy chain variable region having an amino acid sequence shown as SEQ ID No. 1 and a light chain variable region having an amino acid sequence shown as SEQ ID No. 2, wherein the heavy chain variable region and the light chain variable region are connected via a flexible peptide having an amino acid sequence shown as in SEQ ID No. 3.

2. A method for inhibiting tumor growth comprising a step of administrating a subject needed for the inhibiting tumor growth with the humanized anti-VEGFR2 single-chain antibody according to claim 1.

3. The method according to claim 2, wherein the tumor growth is reflected by an increased volume of the tumor and/or an increased mass of the tumor.


SEQUENCE LISTING (6 sequences)

SEQ ID NO: 1 - protein, 118 aa, Artificial Sequence
  It is synthesized.
EVQISESGVPLFQSGASCRGSDAASLTTRSWHEWFAATWARIAYGYGLEGFSGLTFNGAAYSTAESWRGKPTISKENSKNTLYLNFNSIKGEEWGWWYFARAQWFEYWGFGFLSTVSS

SEQ ID NO: 2 - protein, 111 aa, Artificial Sequence
  It is synthesized.
DIQVTNSFATLSLAYADGGTLSCKGSNSWSSQWPAWYNNRPGSGPKLLIWAGSSKGWTGLPEKWAASASGTEFTLTISKIEPDEDSGWWAYCNQPESLGLSFGAGPKVEIK

SEQ ID NO: 3 - protein, 15 aa, Artificial Sequence
  It is synthesized.
GGGGSGGGGSGGGGS

SEQ ID NO: 4 - DNA, 20 bp, Artificial Sequence
  It is synthesized.
ctacggcagccgctggattg

SEQ ID NO: 5 - DNA, 24 bp, Artificial Sequence
  It is synthesized.
ctcgaggcctgaggagacggtgac

SEQ ID NO: 6 - protein, 244 aa, Artificial Sequence
  It is synthesized.
EVQISESGVPLFQSGASCRGSDAASLTTRSWHEWFAATWARIAYGYGLEGFSGLTFNGAAYSTAESWRGKPTISKENSKNTLYLNFNSIKGEEWGWWYFARAQWFEYWGFGFLSTVSSGGGGSGGGGSGGGGSDIQVTNSFATLSLAYADGGTLSCKGSNSWSSQWPAWYNNRPGSGPKLLIWAGSSKGWTGLPEKWAASASGTEFTLTISKIEPDEDSGWWAYCNQPESLGLSFGAGPKVEIK